(19) United States
(12) Patent Application Publication
(10) Pub. No.: US 2019/0054246 A1
(43) Pub. Date: Feb. 21, 2019

(54) MEDICINE DELIVERY DEVICE

(71) Applicant: QUALCOMM Incorporated, San Diego, CA (US)

(72) Inventors: Robert GANTON, San Diego, CA (US); John Earl AMSCHLER, Del Mar, CA (US); Brian NIZNIK, San Diego, CA (US); Eugene DANTSKER, San Diego, CA (US); Paul Robert Hoffman, San Diego, CA (US)

(21) Appl. No.: 16/104,036

(22) Filed: Aug. 16, 2018

Related U.S. Application Data
(60) Provisional application No. 62/547,101, filed on Aug. 17, 2017.

(51) Int. Cl. A61M 5/315

(52) U.S. Cl. CPC A61M 5/31536; A61M 5/31553; A61M 2205/3389; A61M 2005/3126; A61M 2205/50; A61M 2205/0283; A61M 2205/3368

(57) ABSTRACT

Methods, systems, computer-readable media, and apparatuses for facilitating administering of medicine are disclosed.

### RELATED APPLICATION

This patent application claims priority to U.S. Provisional Patent Application Ser. No. 62/547,101, filed Aug. 17, 2017, entitled “MEDICINE DELIVERY DEVICE FOR MITIGATING IMPACT OF AMBIENT CONDITION” which is assigned to the assignee hereof and is incorporated herein by reference in its entirety for all purposes.

### BACKGROUND

Aspects of the disclosure relate to medicine delivering devices, and more particularly to techniques of medicine delivery devices for mitigating the impact of ambient condition.

Certain medical devices may be used to deliver a medicine to a user. An example medical device may be an injection device (e.g., a syringe, an injection pen, etc.). The injection device may hold the medicine in liquid form, and may include a variable dosage setting mechanism (e.g., a piston, dosage ring, limiting mechanism to set piston movement limits, etc.) which allows a user to set a dosage of the medicine to be dispensed. The user may estimate the dosage by reading the numerical scale markings on the injection device. After setting the dosage, the user may operate the injection device to inject the dosage of the medicine into the user's body. The actual dosage set by the user may deviate from the estimated dosage as shown by the numerical scale marking, which in turn affects the proper administering of the medicine.

### SUMMARY

In some embodiments, a medicine delivery device is provided. The medicine delivery device comprises a container and a dosage setting structure. The dosage setting structure is configured to: receive a dosage setting input, and set a volume of space within the container for holding a dosage of a medicine to be delivered based on the dosage setting input. The container, the dosage setting structure, or any combination thereof, is configured such that a first volumetric change of the space matches a second volumetric change of the medicine in the container to within a pre-determined matching tolerance level.

In some aspects, the first volumetric change and the second volumetric change are due to thermal expansion or thermal contraction. The container has a first thermal expansion coefficient. The dosage setting structure has at least a second thermal expansion coefficient. The medicine has a third thermal expansion coefficient. At least one of the first thermal expansion coefficient or the second thermal expansion coefficient is related to the third thermal expansion coefficient such that the first volumetric change and the second volumetric change, both of which are responsive to a change in an ambient temperature within the container, match to within the pre-determined matching tolerance level.

In some aspects, the first thermal expansion coefficient, the second thermal expansion coefficient, the third thermal expansion coefficient, or any combination thereof, is negative.

In some aspects, the dosage setting structure includes a shaft and a piston. The shaft has the second thermal expansion coefficient. The piston has a fourth thermal expansion coefficient of opposite polarity to the second thermal expansion coefficient.

In some aspects, a relationship between an absolute value of the second thermal expansion coefficient and an absolute value of the fourth thermal expansion coefficient reflects a relationship between one or more dimensions of the shaft and the piston such that a volumetric change of the shaft and a volumetric change of the piston, both of which responsive to the change in the ambient temperature within the container, substantially cancel out each other. The one or more dimensions comprise: a length, a width, a cross-sectional area, a volume, or any combination thereof.

In some aspects, the first thermal expansion coefficient and the third thermal expansion coefficient are equal.

In some aspects, the piston and the shaft comprise different materials.

In some aspects, the medicine delivery device further comprises a sensor configured to collect data related to an ambient condition within the container, the data related to the ambient condition comprising a measurement of an ambient temperature, a measurement of an ambient pressure, or any combination thereof. The medicine delivery device further comprises a processing circuit configured to control a volume of the container, the dosage setting structure, or any combination thereof, to set the volume of the space based on the data related to the ambient condition.

In some aspects, the processing circuit is configured to: estimate a volumetric change in the medicine based on the data related to the ambient condition; and control the container, the dosage setting mechanism, or any combination thereof, based on the estimation of the volumetric change.

In some aspects, the processing circuit is configured to adjust a position of a movable component of the dosage setting mechanism with respect to the container to set the volume of the space.

In some aspects, the movable component is coupled with an actuator. The actuator is configured to adjust the position of the movable component based on a signal received from the actuator. In some aspects, the actuator comprises: a piezoelectric device, a shaped memory alloy, an electroactive polymer, or any combination thereof. In some aspects, the actuator is configured to move the movable component by applying an electromagnetic force determined by the processing circuit.

In some aspects, the container comprises an electroactive polymer. The medicine delivery device further includes one or more electrodes. The controlling of the volume of the container comprises the processing circuit being configured to control the one or more electrodes to apply an electric field to the container to induce a volumetric change in the container to set the volume of the space.

In some aspects, the medicine delivery device further comprises a sensor configured to collect data related to an ambient condition within the container, the data related to the ambient condition comprising a measurement of an ambient temperature, a measurement of an ambient pressure, or any combination thereof. The medicine delivery device further comprises a lockable dose dispensing structure for dispensing the medicine. The processing circuit may be configured to determine, based on the data related to the ambient condition, whether the ambient condition exceeds a threshold that defines a safe state of the medicine; and based on a determination of whether the ambient condition exceeds the threshold, lock or unlock the dose dispensing structure.

In some aspects, the dose dispensing mechanism comprises a movable plunger and a latch. The processing circuit is configured to, based on a determination that the ambient condition exceeds the threshold, control the latch to lock the movable plunger at a fixed position to prevent the movable plunger from pushing the medicine from the container.

In some aspects, the dose dispensing mechanism comprises a valve coupled with the container and coupled with the processing circuit. The processing circuit is configured to, based on a determination that the ambient condition exceeds the threshold, control the valve to close to prevent the medicine from leaving the container via the valve.

In some embodiments, a method is provided. The method comprises: receiving, by a dosage setting structure coupled with a container, an input dosage setting, the dosage setting structure and the container being part of a medicine delivery device; and setting, by the dosage setting structure in conjunction with the container, a volume of space within the container for holding a dosage of a medicine to be delivered based on the dosage setting input. A first volumetric change of the space matches a second volumetric change of the medicine in the container responsive to a change in an ambient condition to a pre-determined matching tolerance level.

In some aspects, the first volumetric change and the second volumetric change are due to thermal expansion or thermal contraction. The container has a first thermal expansion coefficient. The dosage setting structure has at least a second thermal expansion coefficient. The medicine has a third thermal expansion coefficient. At least one of the first thermal expansion coefficient or the second thermal expansion coefficient is determined based on the third thermal expansion.

In some aspects, the method further comprises receiving, from a sensor, data related to an ambient condition within the container, the data related to the ambient condition comprising a measurement of an ambient temperature, a measurement of an ambient pressure, or any combination thereof; and setting the volume of the space based on the data related to the ambient condition.

In some aspects, the method further comprises: receiving, from a sensor, data related to an ambient condition within the container, the data related to the ambient condition comprising a measurement of an ambient temperature, a measurement of an ambient pressure, or any combination thereof; determining, based on the data related to the ambient condition, whether the ambient condition exceeds a threshold that defines a safe state of the medicine; and based on determining whether the ambient condition exceeds the threshold, allowing or disallowing delivery of the medicine from the container.

In some embodiments, a non-transitory computer readable medium is provided. The non-transitory computer readable medium stores a set of instructions that, when executed by a hardware processor, causes the hardware processor to perform: receiving, from a dosage setting structure coupled with a container, an input dosage setting, the dosage setting structure and the container being part of a medicine delivery device; and controlling, the dosage setting structure, the container, or any combination thereof, a volume of space within the container for holding a dosage of a medicine to be delivered based on the dosage setting input. A first volumetric change of the space matches a second volumetric change of the medicine in the container responsive to a change in an ambient condition to a pre-determined matching tolerance level.

In some embodiments, an apparatus is provided. The apparatus comprises: means for holding a medicine; means for receiving a dosage setting input; and means for setting a volume of space within the means for holding the medicine, the volume of space for holding a dosage of a medicine to be delivered based on the means for receiving the dosage setting input. The means for holding the medicine, the means for receiving the dosage setting input, or any combination thereof, is configured such that a first volumetric change of the space matches a second volumetric change of the medicine to within a pre-determined matching tolerance level.

### BRIEF DESCRIPTION OF DRAWINGS

Non-limiting and non-exhaustive aspects are described with reference to the following figures, wherein like reference numerals refer to like parts throughout the various figures unless otherwise specified.

FIG. 1 illustrates a simplified diagram of a system for providing information about the administration of medicine by a medicine delivery device;

FIG. 2A and FIG. 2B illustrate simplified diagrams of an example medicine delivery device, according to certain aspects of the present disclosure;

FIG. 3 illustrates a simplified block diagram of example components of the medicine delivery device of FIG. 2A and FIG. 2B;

FIG. 4 illustrates a simplified diagram of the internal structure of the medicine delivery device of FIG. 2A and FIG. 2B;

FIG. 5 illustrates an example of a dosage error caused by ambient conditions which may be mitigated by aspects of the present disclosure;

FIGS. 6-10 illustrate examples of components of a medicine delivery device for mitigating the impact of ambient conditions, according to certain aspects of the present disclosure; and

FIG. 11A, FIG. 11B, and FIG. 11C illustrate examples of methods to facilitate administering of medicine, according to certain aspects of the present disclosure.

### DETAILED DESCRIPTION

Several illustrative examples will now be described with respect to the accompanying drawings, which form a part hereof. While particular examples, in which one or more aspects of the disclosure may be implemented, are described below, other examples may be used and various modifications may be made without departing from the scope of the disclosure or the spirit of the appended claims.

A medicine delivery device, such as an injection pen, enables administering of a pre-set dosage of medicine to a user. The medicine delivery may hold the medicine in liquid form, and includes a variable dosage setting mechanism (e.g., a piston, dosage ring, limiting mechanism to set piston movement limits, etc.) which allows a user to set a dosage of the medicine to be dispensed. The user may estimate the dosage by reading the numerical scale markings on the injection device. After setting the dosage, the user may operate the injection device to inject the dosage of the medicine into the user's body.

Although a medicine delivery device, such as an injection device, provides an effective way of administering a medicine to a user, the actual dosage set by the user may deviate from the estimated dosage as shown by the numerical scale marking, which in turn affects the proper administering of the medicine. First, both the medicine and the injection device may undergo thermal expansion or contraction based on the ambient temperature and (to a lesser extent) the ambient pressure. The volumetric changes of the medicine and the injection device may introduce dosage error, such that the actual dosage administered is not the same as the dosage as shown by the numerical scale marking. For example, due to heating or reduced pressure, a medicine in liquid form may expand. The container of the injection device that holds the medicine may also expand at a different rate from the medicine. As a result, a certain volume (e.g., 10 mL) of the medicine indicated by the scale markings of the container may contain different quantities of molecules at different temperatures, which may lead to different dosages of the medicine being administered at different temperatures. Second, the chemical properties of the medicine may change when the ambient temperature exceeds a certain threshold, which may reduce the medicine's effectiveness or even render the medicine unsafe. Without accounting for the ambient conditions, an incorrect dosage of medicine, or a dose of perished medicine, may be administered to the user, which may have adverse effects on the user.

Disclosed are techniques to mitigate effects of ambient conditions on the administering of medicine. In some embodiments, a medicine delivery device is provided, the medicine delivery device including a container and a dosage setting structure. The dosage setting structure may receive a dosage setting input, and set a volume of space (hereinafter, “set volume”) within the container for holding a dosage of the medicine to be delivered based on the dosage setting input. The container, the dosage setting structure, or any combination thereof, may be configured such that a first volumetric change of the space matches a second volumetric change of the medicine in the container to a pre-determined matching tolerance level.

In some embodiments, the first volumetric change and the second volumetric change may be caused by thermal expansion or thermal contraction responsive to a change in an ambient temperature within the container. The container may have a first thermal expansion coefficient. The dosage setting structure may have at least a second thermal expansion coefficient. The medicine may have a third thermal expansion coefficient. The first thermal expansion coefficient of the container and/or the second thermal expansion coefficient of the dosage setting structure may be determined based on the third thermal expansion coefficient of the medicine. For example, the first thermal expansion coefficient of the container and/or the second thermal expansion coefficient of the dosage setting structure may be determined such that a first rate of thermal expansion or contraction of the space matches a second rate of thermal expansion or contraction of the medicine.

In some embodiments, the medicine delivery device may include a sensor configured to collect data related to an ambient condition within the container, the data related to the ambient condition comprising a measurement of an ambient temperature, a measurement of an ambient pressure, or any combination thereof. The sensor may be configured to collected the data from an ambient environment within the container, from an environment external to the container, and/or from the medicine held in the container directly. The medicine delivery device may further include a processing circuit configured to control the container, the dosage setting mechanism, or any combination thereof, to set the volume of the space based on the data related to the ambient condition. In some embodiments, the dosage setting mechanism may include an actuator which may be controlled by the processing circuit to adjust the set volume based on the sensor data. In some embodiments, the container may comprise an electroactive polymer which may expand or contract based on an electric field from one or more electrodes, and the processing circuit may control the electric field based on the sensor data to adjust a volume of the container.

In some embodiments, the medicine delivery device may further include a lockable dose dispensing mechanism for dispensing the medicine. The processing circuit may determine, based on the sensor data, whether the ambient condition exceeds a threshold that defines a safe state of the medicine. The processing circuit may lock the dose dispensing mechanism to disallow delivery of the medicine if the ambient condition exceeds the threshold, which may indicate that the medicine is likely to be in an unsafe state. The processing circuit may unlock the dose dispensing mechanism to disallow delivery of the medicine if the ambient condition does not exceed the threshold, which may indicate that the medicine is likely to be in a safe state.

With the disclosed techniques, a medicine delivery device may be configured to adjust the administering of medicine (e.g., by adjusting a dosage of the medicine, to disable the administering of the medicine, etc.) to account for the impact of the ambient environment on a physical state (e.g., volume, chemical composition, etc.) of the medicine. Dosage setting may become more accurate by compensating for dosage errors introduced by volumetric change of the medicine as well as the medicine delivery device caused by the ambient environment. Moreover, medicines that are rendered potentially unsafe/ineffective by the change in the ambient environment may also be screened. All of these may facilitate proper administering of the medicine to a user and may reduce health hazard posed by improper administering of the medicine (e.g., administering incorrect dosage of the medicine, administering the medicine when the medicine has been perished, etc.).

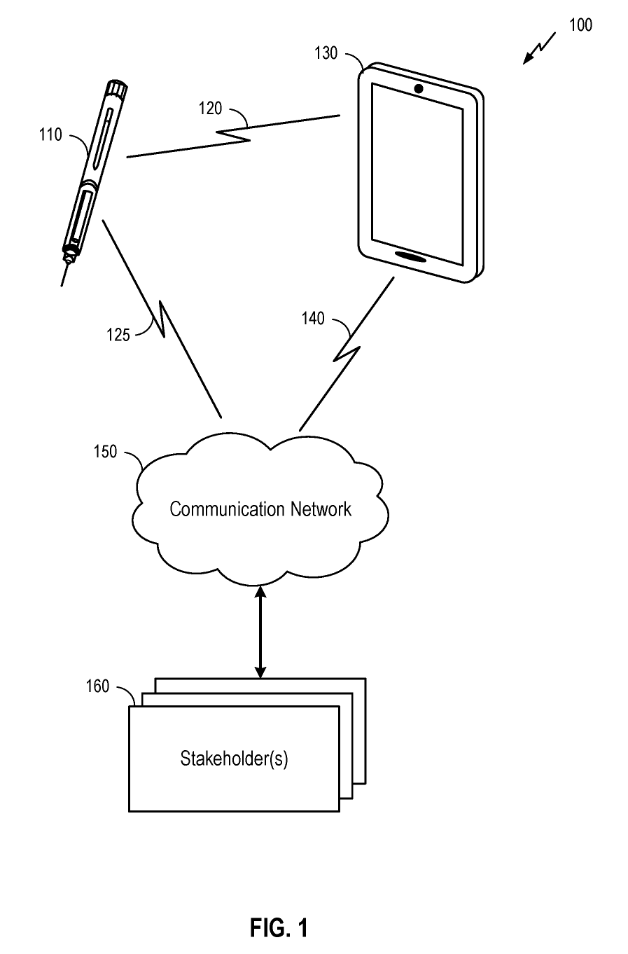

FIG. 1 illustrates a system 100 for providing information about the administration of medicine by a medicine delivery device 110 to one or more stakeholder(s) 160. Here, the system 100 may comprise medicine delivery device 110 as described herein, along with a connecting device 130, communication network 150, and the stakeholder(s) 160. Medical delivery device 110 may be a device configured to deliver medicine in liquid form. It will be understood, however, that examples of a system 100 may include a different configuration of components, the addition and/or omission of various components, and/or the like, depending on desired functionality. Moreover, it will be understood that techniques described herein may be utilized in a medicine delivery device 110 that may not necessarily be part of a larger system, such as the system 100 illustrated in FIG. 1.

Medicine delivery device 110, which is described in more detail herein below, may be used to administer a medicine to a patient. In FIG. 1, medicine delivery device 110 may be an injection device such as, for example, a syringe, an injection pen, etc. Here, a person (e.g., a doctor, nurse, or patient him/herself) may administer the medicine by engaging a physical mechanism (e.g., pressing down on a plunger, actuating automatic injection, etc.). Through the physical mechanism, a dose of the medicine may be injected into the patient's skin via a needle of medicine delivery device 110 inserted into the patient's skin. In some embodiments, once the medicine is administered, medicine delivery device 110 may then register, store and transmit data associated with the administration of the medicine to connecting device 130. This data may be transmitted wirelessly via a wireless communication link 120, using any of a variety of wireless technologies as described in further detail below. That said, some examples may additionally or alternatively utilize wired communication.

Connecting device 130 may comprise any of a variety of electronic devices capable of receiving information from medicine delivery device 110 and communicating information to the stakeholder(s) 160 via communication network 150. This may include, for example, a mobile phone, tablet, laptop, portable media player, personal computer, or similar device. In some embodiments, connecting device 130 may comprise a specialized device utilized for purposes of conveying information from medicine delivery device 110 (and possibly other medical devices) to stakeholder(s) 160. In some embodiments, the connecting device 130 may comprise a device owned and operated by the patient (e.g., the patient's mobile phone). In other embodiments, the connecting device 130 may be owned and/or operated by another entity, such as a healthcare provider, insurance company, government agency, etc.

Connecting device 130 may execute an application to provide the data processing and/or relaying functionality illustrated in FIG. 1. In some embodiments, the application may be configurable by a user, or may simply be downloaded to connecting device 130 and executed automatically. The application may help establish communication link 120 between medicine delivery device 110 and the connecting device 130, which may or may not require input from the user, depending on desired functionality. In some embodiments, the application may provide instructions to a user on proper use of medicine delivery device 110 and/or feedback to a user about the detected use of medicine delivery device 110. As to be discussed in more detail below, medicine delivery device 110 may detect a dosage set by the patient, and transmit information related to the detected dosage to connecting device 130 (e.g., whether incorrect dosage is set), as part of the feedback. Additional and/or alternative functionality of an application executed by the connecting device 130 may be utilized as desired such as, for example, relaying of the data to a remote destination, interacting with the patient about the medicine administration, etc.

Communication network 150 may comprise any of a variety of data communication networks, depending on desired functionality. Communication network 150 may include any combination of radio frequency (RF), optical fiber, satellite, and/or other wireless and/or wired communication technologies. In some embodiments, communication network 150 may comprise the Internet and/or different data networks may comprise various network types, including cellular networks, Wi-Fi® networks, etc. These types may include, for example, a Code Division Multiple Access (CDMA) network, a Time Division Multiple Access (TDMA) network, a Frequency Division Multiple Access (FDMA) network, an Orthogonal Frequency Division Multiple Access (OFDMA) network, a Single-Carrier Frequency Division Multiple Access (SC-FDMA) network, a WiMax (IEEE 802.16), and so on. A CDMA network may implement one or more radio access technologies (RATs) such as cdma2000, Wideband-CDMA (W-CDMA), and so on. Cdma2000 includes IS-95, IS-2000, and/or IS-856 standards. A TDMA network may implement Global System for Mobile Communications (GSM), Digital Advanced Mobile Phone System (D-AMPS), or some other RAT. An OFDMA network may employ LTE (including LTE category M (CatM) or 5G), LTE Advanced, and so on. LTE, LTE Advanced, GSM, and W-CDMA are described in documents from 3GPP. Cdma2000 is described in documents from a consortium named “3rd Generation Partnership Project 2” (3GPP2). 3GPP and 3GPP2 documents are publicly available. The communication network 150 may additionally or alternatively include a wireless local area network (WLAN), which may also be an IEEE 802.11x network, and a wireless personal area network (WPAN) may be a Bluetooth network, an IEEE 802.15x, Zigbee® network, and/or some other type of network. The techniques described herein may also be used for any combination of wireless wide area network (WWAN), WLAN and/or WPAN.

Communication link 140 between connecting device 130 and communication network 150 may vary, depending on the technologies utilized by these components of the system 100. For examples where connecting device 130 is a smart phone capable of connecting with a cellular network and/or a Wi-Fi® network, communication link 140 may comprise a wireless communication link utilizing the mobile phone's cellular or Wi-Fi® functionality. In examples where connecting device 130 is a personal computer, communication link 140 may comprise a wired communication link that accesses communication network 150 via a cable or digital subscriber line (DSL) modem.

It may be noted that some embodiments may not utilize a connecting device 130 to relay data to the communication network 150. In such embodiments, medicine delivery device 110 may connect directly to communication network 150 (as shown in FIG. 1 by communication link 125, which may be used in addition to or as an alternative to communication link 120). For example, medicine delivery device 110 may comprise a Long Term Evolution (LTE) category M (CatM) device, NarrowBand IoT (NB-IoT), or other Low Power Wide Area Network (LPWAN). Additionally or alternatively, medicine delivery device 110 may comprise wireless technology similar to the corresponding functionality of connecting device 130 described above. In these embodiments, the communication network may additionally or alternatively comprise a Bluetooth Mesh network (such as CSRMesh), a WiFi network, Zigbee, or WWAN (such as LTE, including CATM, or 5G). In some embodiments, medicine delivery device 110 may connect both with communication network 150 via communication link 125 and with connecting device 130 via communication link 120. In such embodiments, the connecting device 130 may not need to separately communicate information regarding medicine delivery device 110 to stakeholders 160, but instead medicine delivery device 110 may communicate this information directly to the stakeholders 160 via the communication network 150.

As noted above, the stakeholder(s) 160 may include any of a variety of entities with an interest in the proper administration of medicine by medicine delivery device 110. This may include an individual practitioner (e.g., a doctor or nurse), a hospital, a drug manufacturer, an insurance provider (or other payer), a government agency or other health organization, and/or the like. In some embodiments, the user of medicine delivery device 110 (e.g., the patient) may also be a stakeholder 160 to which information regarding the use of medicine delivery device 110 is provided. Governmental health regulations and/or legal agreements between the patient and/or stakeholder(s) 160 may apply to the dissemination of information regarding the administration of a drug by medicine delivery device 110 to stakeholder(s) 160. Here, as mentioned above and described in further detail below, medicine delivery device 110 may detect a dosage set by the patient, and transmit information related to the detected dosage to stakeholder(s) 160 (e.g., the patient operating connecting device 130, or other people via other devices).

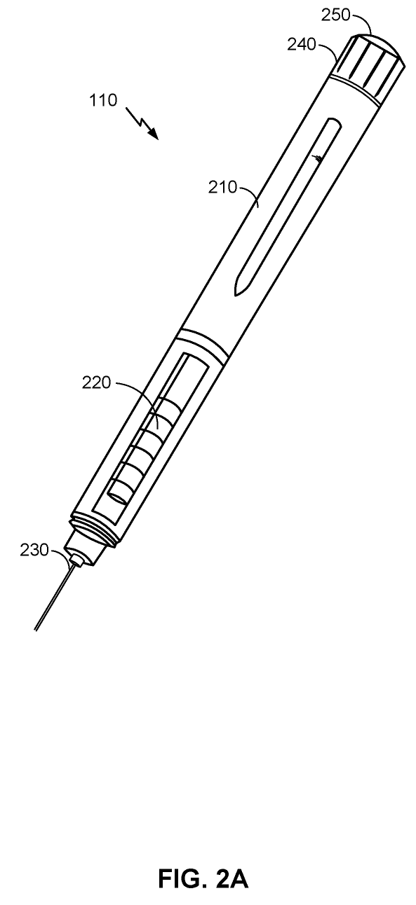

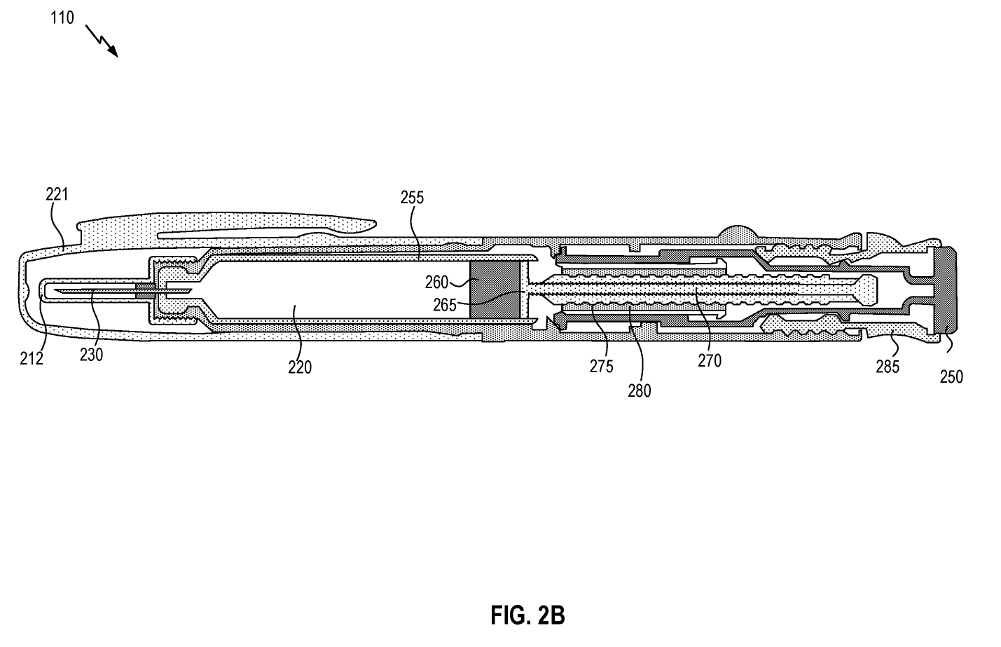

FIG. 2A is an illustration of an example of medicine delivery device 110, according to certain aspects of the present disclosure. Here, a body 210 of medicine delivery device 110 may provide a housing for dose dispensing and dose control mechanisms. The dose control mechanisms may include one or more electronic and mechanical components for setting a dosage of medicine to be administered. Moreover, the dose dispensing mechanism may include one or more electronic and mechanical components for dispensing the dosage of medicine. For example, the mechanical components of a dose dispensing mechanism may include a movable component (e.g., a piston) controlled by the dose control mechanism and configured to displace a volume of the medicine through the reservoir chamber 220 and out of needle assembly 230. Medicine delivery device 110 further includes a dose knob 240 that may be adjusted (e.g., by turning the knob clockwise or counterclockwise) to alter the dosage to be administered by medicine delivery device 110. The dosage may be administered by pressing dose dispensing button 250, which may be coupled to a dose dispensing mechanism to control the dispensing of the drug.

FIG. 2B is a cross-sectional view of medicine delivery device 110, according to an embodiment. In the illustration in FIG. 2B, some components not illustrated in FIG. 2A are shown, including the pen cap 221 and inner needle cap 212. The pen injector further includes a cartridge 255 that stores the medicine and comprises reservoir chamber 220. (In some embodiments, the cartridge may be replaceable, enabling medicine delivery device 110 to be used with multiple cartridges.) During the administration of the drug, a piston 260 is pushed by head 265 of a drive stem 270, displacing the drug in reservoir chamber 220 to administer the drug. Drive stem 270 may be screw driven, having threads 275 that feed drive stem 270 through a nut 280. When the user presses dose dispensing button 250, the medicine in reservoir chamber 220 may be displaced via movement of drive stem 270 and piston 260. A volume of space within reservoir chamber 220 for holding the medicine to be displaced may be regulated by dose selector 285.

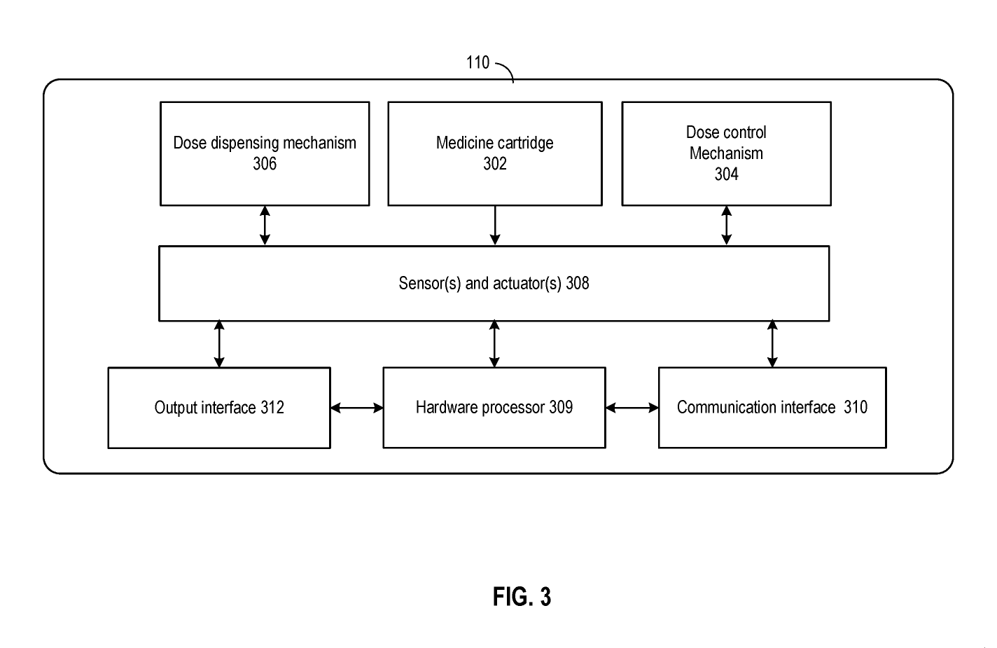

It will be understood, however, that medicine delivery device 110 illustrated in FIG. 2A and FIG. 2B is provided as a non-limiting example, according to an example. Alternative examples may vary in size, shape, and/or other ways. A medicine delivery device 110 may be described more generally as having various components as illustrated in FIG. 3.

FIG. 3 is a block diagram illustrating example components of medicine delivery device 110, according to certain aspects of the present disclosure. Medicine delivery device 110 may include a housing (not shown) structured to hold a medicine cartridge 302, which may store medicine to be dispensed by medicine delivery device 110. Medicine delivery device 110 may also include a dose control mechanism 304 to select or set a dosage of the medicine to be dispensed. For example, dose control mechanism 304 may include a piston to set a volume of medicine held within medicine cartridge to be administered. Medicine delivery device 110 further includes a dose dispensing mechanism 306 to dispense a dose of the drug, from medicine cartridge 302, based on the dosage selected or set by dose control mechanism 304.

Medicine delivery device 110 may include other devices to facilitate administering of medicine. In the example of FIG. 3, medicine delivery device 110 includes sensor(s) and actuator(s) 308 and a hardware processor 309. Sensor(s) and actuator(s) 308 may include sensors and actuators to control the operations of the actuators based on the information collected by the sensor. For example, the sensors of sensor(s) and actuator(s) 308 may collect information of certain physical conditions at, for example, medicine cartridge 302, dose control mechanism 304, and dose dispensing mechanism 306. Based on the collected information, hardware processor 309 may include hardware circuit configured to control the actuators of sensor(s) and actuator(s) 308 to change the operations of dose control mechanism 304 and/or dose dispensing mechanism 306. For example, as to be described in more detail below, the sensors of sensor(s) and actuator(s) 308 may generate data related to one or more ambient conditions (e.g., temperature, pressure, etc.) inside medicine cartridge 302. Based on the ambient conditions, hardware processor 309 may configure dose control mechanism 304 to set a dosage to account for volumetric change of the medicine stored in medicine cartridge 302, as well as volumetric change of medicine cartridge 302 as well as dose control mechanism 304, both of which may be caused by the one or more ambient conditions. Moreover, hardware processor 309 may also configure dose dispensing mechanism 306 to, for example, prevent dispensing of the medicine from medicine cartridge 302, based on a determination that the temperature in the cartridge is high enough to render the medicine unsuitable for consumption.

Moreover, medicine delivery device 110 may include a communication interface 310 and an output interface 312. Communication interface 310 may communicate using wireless and/or wired means (e.g., via wireless communication link 120 and/or 125 of FIG. 1). Communication interface 310 may enable transmission of information related to administering the drug. For example, communication interface 310 may enable transmission of information indicating a dosage set by the user and, in the event that an incorrect dosage is set, may enable transmission of a warning to the user about the incorrect dosage. The information may then be displayed to the user via an user interface, to assist the user in administering of the medicine. Moreover, as part of an interactive process, communication interface 310 may also receive information related to a confirmation (or an overriding command) from the user that the medicine is to be administered according to the set dosage. Communication interface 310 may relay the confirmation or overriding command to sensor(s) and actuator(s) 308, to enable dose dispensing mechanism 306 to dispense the medicine.

On the other hand, output interface 312 may be controlled by hardware processor 309 to output operation information of medicine delivery device 110. For example, output interface 312 may output (e.g., in display form, in audio form, etc.) a dosage set by the user. The output dosage information may be adjusted based on the configuration of dose control mechanism 304, to accounting for volumetric changes of the medicine and of medicine cartridge 302 as well as dose control mechanism 304. The user may then use the output dosage information as a guide to set the dosage. Output interface 312 may include, for example, a display interface and/or an audio device (e.g., to display or speak out a set dosage), a mechanical device (e.g., to produce a click sound whenever a pre-determined unit dosage is selected and is added or subtracted from an initial dosage), etc.

Although not shown in FIG. 3, medicine delivery device 110 may further include one or more non-transitory storage devices including, for example, a solid-state storage device, such as a random access memory (“RAM”), and/or a read-only memory (“ROM”), which may be programmable, flash-updateable and/or the like. Such storage devices may be configured to implement any appropriate data stores, including without limitation, various file systems, database structures, and/or the like. A set of these instructions and/or code might be stored on a non-transitory computer-readable storage medium, which may then be executed by hardware processor 309 to perform the operations described above and operations to be described below.

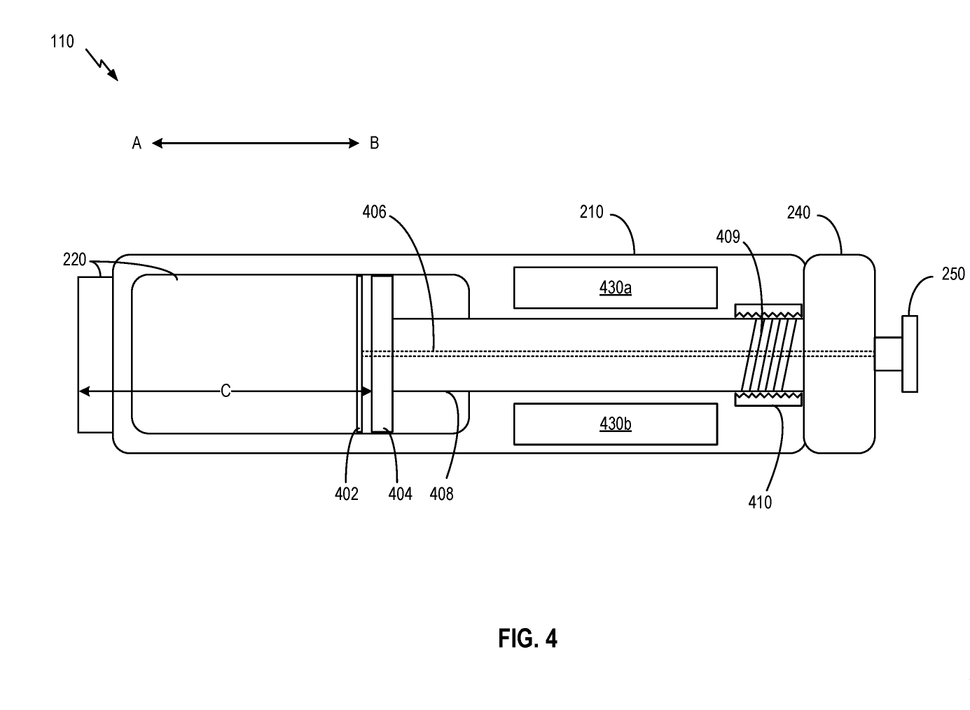

FIG. 4 is a simplified illustration of an internal structure of medicine delivery device 110, according to an example. It will be appreciated by a person of ordinary skill in the art that the illustration is not to scale, and the various components illustrated may vary in size, shape, arrangement, and/or other ways, as desired. As shown in FIG. 4, medicine delivery device 110 may include a dose dispensing piston 402 and a dose setting piston 404 which may correspond to, respectively, piston 260 and head 265 of FIG. 2B. Dose dispensing piston 402 is coupled with dose dispensing button 250 via a shaft 406, whereas dose setting piston 404 is coupled with dose knob 240 via a screw shaft 408. Shaft 406 may correspond to drive stem 270 of FIG. 2B. For example, screw shaft 408 may include a set of helical grooves 409 coupled with body 210 via screw receiver 410. As screw shaft 408 rotates, and guided by helical grooves 409 and screw receiver 410, screw shaft 408 may also perform a linear motion. For example, as a user rotates dose knob 240 in one direction (e.g., a clockwise direction or a counter-clockwise direction), screw shaft 408 may push dose setting piston 404 towards direction A, or pull dose setting piston 404 towards direction B. The distance between dose setting piston 404 and the left edge of reservoir chamber 220 (indicated by a reading of distance C on medicine delivery device 110) may set a volume of a space within reservoir chamber 220 (e.g., a “set volume”) to hold the medicine to be dispensed, which may correspond to the dosage selected by the user. After the user finishes rotating dose knob 240 to set a dosage based on the set volume reading indicated by distance C, the user may push down dose dispensing button 250, which then pushes dose dispensing piston 402 towards direction A to push the medicine out of reservoir chamber 220. In some embodiments, dose dispensing piston 402, dose setting piston 404, shaft 406, screw shaft 408, and dose dispensing button 250 may be part of a plunger. Dose setting piston 404, screw shaft 408, and dose knob 240 may be part of dose control mechanism 304 of FIG. 3, whereas dose dispensing piston 402, shaft 406, and dose dispensing button 250 may be part of dose dispensing mechanism 306. Dose knob 240, screw shaft 408, helical grooves 409, and screw receiver 410 may correspond to (or have similar functions as) dose selector 285 and nut 280 of FIG. 2B.

In some embodiments, as to be discussed in detail below, reservoir chamber 220, dose dispensing piston 402, dose setting piston 404, shaft 406, screw shaft 408, or any combination thereof, may be configured such that a thermal expansion rate of the set volume may be approximately the same as the medicine held in reservoir chamber 220. With such arrangements, a set volume within reservoir chamber 220 that holds the medicine to be dispensed, which may be defined by the distance C of FIG. 4 and the cross-sectional area of reservoir chamber 220, may expand or contract based on the ambient temperature within reservoir chamber 220 (which also reflects the temperature of the medicine), such that the set volume has the same thermal expansion rate as the medicine. With such arrangements, the quantity of molecules in a dosage of the medicine having a particular set volume reading (e.g., based on a reading of distance C on medicine delivery device 110) may be substantially the same across different temperatures, and a correct dosage of the medicine may be administered across different temperatures.

In some embodiments, as to be discussed in detail below, the set volume may also be adjusted by electronic circuitries. For example, body 210 may hold electronic units 430a and 430b, which may include sensors and actuators of sensor(s) and actuator(s) 308, hardware processor 309, communication interface 310, output interface 312, etc. The sensors of electronic units 430a and 430b may include sensors (e.g., temperature sensors, pressure sensors, etc.) to sense the ambient conditions within reservoir chamber 220. Based on the ambient conditions, a processing circuit (e.g., hardware processor 309) may adjust a position of dose setting piston 404 within reservoir chamber 220, which in turn may adjust the set volume within reservoir chamber 220 to hold the medicine to be administered. The adjustment of the position of dose setting piston 404 may be based the detected ambient conditions (e.g., the ambient temperature, the ambient pressure, etc.) to track the expansion (or contraction) of the medicine in the reservoir chamber. With such arrangements, the quantity of molecules in a dosage of the medicine having a particular set volume reading (e.g., based on a reading of distance C on medicine delivery device 110) may be substantially the same across different temperatures and pressures, to ensure administering a correct dosage of the medicine. The adjustment of the position of dose setting piston 404 may be performed by, for example, moving screw shaft 408, screw receiver 410, or any combination thereof relative to reservoir chamber 220.

Alternatively or in combination, the processing circuit may also adjust the size of reservoir chamber 220 to adjust the set volume. For example, reservoir chamber 220 may be made of an electroactive polymer and may expand or contract when subject to an electric field. To control the electroactive polymer, medicine delivery device 110 may include a pair of electrodes (not shown in FIG. 4) surrounding reservoir chamber 220, and the pair of electrodes may be controlled by the processing circuit to set an electric field to change the size of reservoir chamber 220. The strength of the electric field may be set based on the detected ambient conditions, such that the size of reservoir chamber 220 and the set volume defined by distance C may be adjusted based on the detected ambient conditions as well. With such arrangements, the quantity of molecules in a dosage of the medicine having a particular set volume reading (e.g., based on a reading of distance C on medicine delivery device 110) may be substantially the same across different temperatures and pressures, to ensure administering a correct dosage of the medicine.

In some embodiments, electronic units 430a and 430b may also include actuators to lock dose dispensing piston 402 (and/or shaft 406) at a fixed position to prevent dose dispensing piston 402 from pushing the medicine out of reservoir chamber 220. The locking may occur under various circumstances. For example, electronic units 430a and 430b may lock dose dispensing piston 402 (and/or shaft 406) at a fixed position in the event that the ambient temperature inside reservoir chamber 220 exceeds a threshold associated with a safe state of the medicine. As another example, electronic units 430a and 430b may implement a security mechanism to authenticate a user who tries to use medicine delivery device 110 to administer a medicine. If the authentication fails, electronic units 430a and 430b may also lock dose dispensing piston 402 (and/or shaft 406) at a fixed position.

Moreover, electronic units 430a and 430b may include other circuitries, such as communication interface circuitries of communication interface 310, as well as circuitries of output interface 312. The communication interface circuitries may transmit information about the dosage set, and receive a confirmation or overriding command to release the locking of dose dispensing piston 402 (and/or shaft 406), as described above. The circuitries of output interface 312 may include circuitries for outputting dosage information (e.g., in display form, in audio form, etc.)

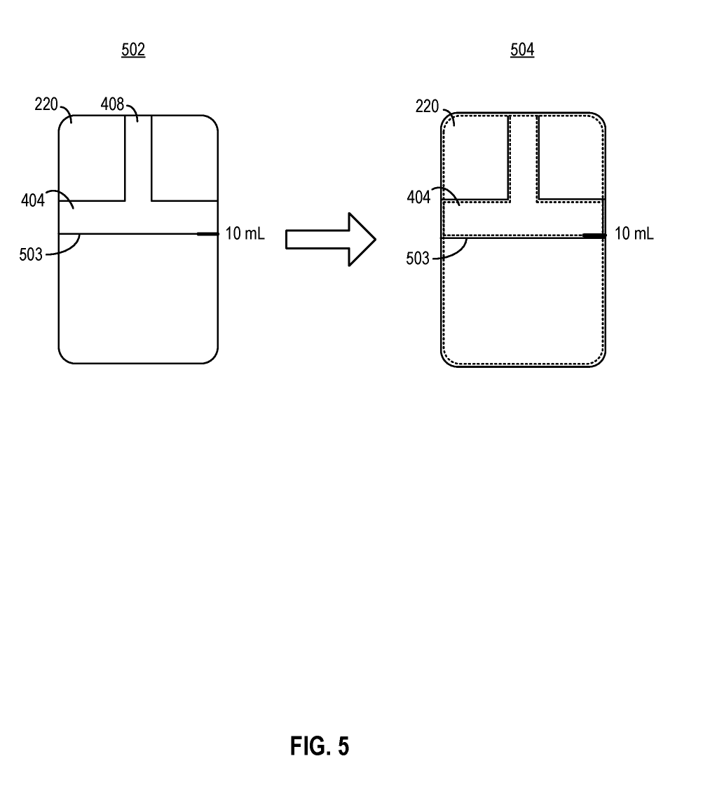

FIG. 5 illustrates examples of a dosage error caused by the ambient conditions. Assuming that at time 502, at a temperature of 65 F, a dosage of a medicine corresponding to 10 mL is set. The 10 mL dosage is set based on an alignment of level 503 of the medicine with a 10 mL scale marking in reservoir chamber 220, and based on positioning (with screw shaft 408) dose setting piston 404 at the scale marking, which defines a set volume within reservoir chamber 220 to hold 10 mL of the medicine. At time 504, at a raised temperature of 85 F, the medicine, as well as reservoir chamber 220, dose setting piston 404, and screw shaft 408 may expand by an amount based on their associated thermal expansion coefficients and dimensions, with the dotted lines representing the dimension of reservoir chamber 220, dose setting piston 404, and screw shaft 408 at time 502. In the example of FIG. 5, the combined expansions may result in, for example, level 503 of the medicine falling below the 10 mL scale marking in reservoir chamber 220, even though there is no change in the quantity of the molecules in the medicine below dose setting piston 404. A user, who is unaware of the volumetric changes of the medicine and of medicine delivery device 110 due to the change in temperature, may believe that there is insufficient dosage based on level 503 of the medicine falling below the 10 mL scale marking, and may adjust dose setting piston 404 to bring it to align with the 10 mL scale marking to add in more medicine to be administered. As a larger number of medicine molecules are included in the dosage, overdose may result.

Besides thermal expansion, the volume of the medicine may also change due to ambient atmospheric pressure inside reservoir chamber 220. For example, assuming that reservoir chamber 220 contains a mixture of the medicine and air, the atmospheric pressure may change based on a location of the medicine delivery device, which may change the volume of the medicine. Referring back to FIG. 5, a user may operate medicine delivery device 110 at a low altitude (where the air pressure is relatively high) at time 502, and the user may rely on the alignment of level 503 with the 10 mL scale marking as an indication that a volume of 10 mL is set. If the user then operate medicine delivery device 110 at a high altitude (where the air pressure is relative low) at time 504, the medicine may expand such that medicine molecules may escape from the set volume and escape to the part of reservoir chamber 220 at the back of dose setting piston 404, and the quantity of medicine molecules administered may reduce, which may result in under-dose.

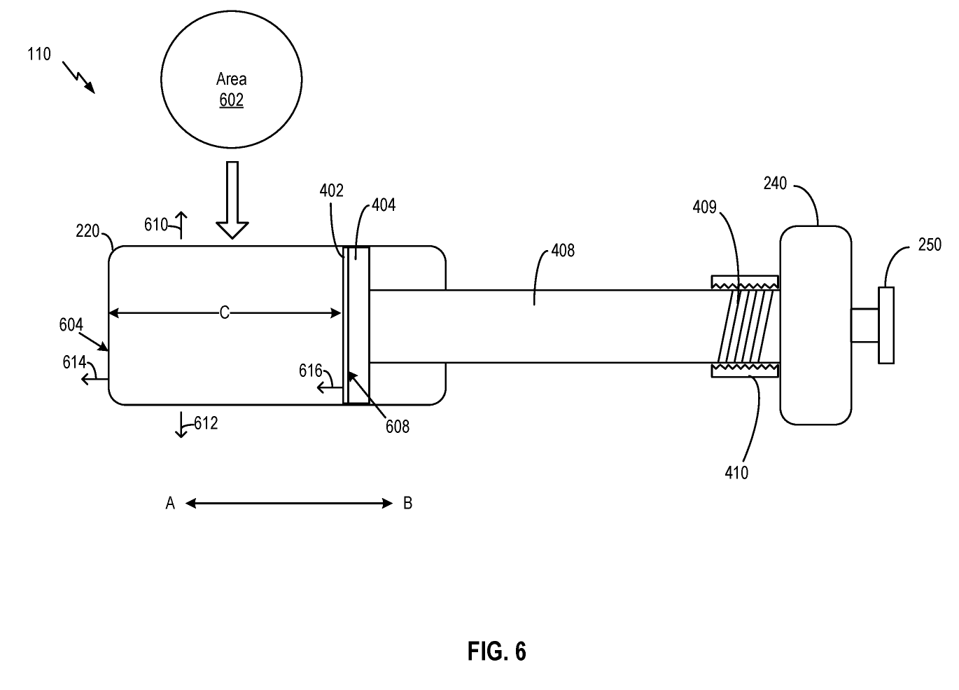

FIG. 6 illustrates an embodiment of medicine delivery device 110 that may operate to mitigate the impact of ambient temperature, according to certain aspects of the present disclosure. For illustration purposes, only some of the components of medicine delivery device 110 are shown in FIG. 6. As discussed above, one way to mitigate the impact of thermal expansion (or contraction) is to have a combined thermal expansion rate of a set volume defined by reservoir chamber 220, dose dispensing piston 402, dose setting piston 404, and screw shaft 408 to match (to within a pre-determined matching tolerance level) the thermal expansion rate of the medicine, such that the set volume tracks the actual volume of the medicine across different temperatures. The matching tolerance level can be, for example, a preset percentage of a volume of the reservoir chamber 220, a preset percentage of the dosage to be set, etc. In FIG. 6, the set volume may be defined as a product of the cross-sectional area 602 of reservoir chamber 220, and the distance C. To have the set volume to track the volume of the medicine, the contributions to the thermal expansion rate of the set volume by the thermal expansion rates of reservoir chamber 220, dose dispensing piston 402, dose setting piston 404, and screw shaft 408 may be determined. The combined thermal expansion rate of the set volume may be determined based on a weighted average of the thermal expansion rates of reservoir chamber 220, dose dispensing piston 402, dose setting piston 404, and screw shaft 408. The weights may be determined based on, for example, the dimensions of each component. The weights may also be positive or negative based on whether an expansion of a component increases or reduces the set volume.

For example, referring to FIG. 6, the distance C is defined between the left edge 604 of reservoir chamber 220 and the left edge 608 of dose setting piston 404 (and dose dispensing piston 402). As reservoir chamber 220 expands, cross-sectional area 602 increases and expands (as shown by arrows 610 and 612), which increases the set volume. Also, as shown by the arrows 614 and 616 in FIG. 6, the left edge 604 of reservoir chamber 220 also moves to the left (towards direction A), which tends to increase distance C. On the other hand, as dose dispensing piston 402, dose setting piston 404, and screw shaft 408 expand, left edge 608 of dose setting piston 404 (and dose dispensing piston 402) moves to the left (towards direction A), which may offset some of the gains in the volume by expansion of left edge 604 to the left. Accordingly, the thermal expansion of reservoir chamber 220 leads to expansion of the set volume, while the thermal expansions of dose dispensing piston 402, dose setting piston 404, and screw shaft 408 may lead to contraction of the set volume. Moreover, each of these components may have a different thermal expansion rate, which leads to the components contributing to the thermal expansion of the set volume by different degrees. The different thermal expansion rates may be attributed to different thermal expansion coefficients and dimensions of the components.

In order to provide substantially the same thermal expansion rate for the set volume and for the medicine, at least one of the dimension or the thermal expansion coefficient of each component may be determined to set a thermal expansion rate for each component. Moreover, each of the thermal expansion rate may be scaled by a weight of which the sign and the value may be determined based on, for example, a relationship between the dimension of the component and the dimension of the set volume, whether the component and the set volume expand in the same direction or in opposite direction with temperature, etc. The following is an illustrative example. Assuming both the medicine, reservoir chamber 220, and piston 402 expand with temperature, all of which having positive thermal expansion coefficients. While the expansion of reservoir chamber 220 increases the set volume, the expansion of piston 402 leads to reduction of the set volume. To compensate for the set volume reduction effect from piston 402, screw shaft 408 may be made to contract when temperature increases (e.g., having a negative thermal expansion coefficient, or otherwise an opposite polarity of thermal expansion coefficient to the coefficient of piston 402). The absolute values of the thermal expansion coefficients of screw shaft 408 may be determined based on a relationship between certain dimensions (e.g., a length, a width, a cross-sectional area, a volume, etc.) of screw shaft 408, reservoir chamber 220, and piston 402. In this particular example, the medicine's thermal expansion coefficient is 0.1/C, the reservoir's thermal expansion coefficient is 0.1/C, and the piston's thermal expansion coefficient is 0.5/C. To compensate for the thermal expansion of piston 402 (which would reduce the thermal expansion of the set volume by the reservoir), the thermal expansion coefficient of screw shaft 408 may be chosen based on a ratio between the length of screw shaft 408 and the length of piston 402. For example, assuming that the length of screw shaft 408 is ten times of the thickness of piston 402, the thermal expansion coefficient of screw shaft 408 may be −0.05/C (one-tenth of the thermal expansion coefficient of the piston) such that the thermal contraction of screw shaft 408 may cancel out the thermal expansion of piston 402, and the set volume may expand at the same rate as the medicine based on reservoir chamber 220 having the same thermal expansion coefficient and dimension as the medicine held within the chamber. In another example, piston 402 may have negative thermal expansion coefficient, whereas screw shaft 408 may have positive thermal expansion coefficient. The thermal expansion coefficient of screw shaft 408 may also be determined based on ratio between the length of screw shaft 408 and the length of piston 402. In yet another example, the reservoir's thermal expansion rate may be different from the medicine's thermal expansion rate (e.g., due to different thermal expansion coefficients, dimension, or any combination thereof). In this case, the combined thermal expansion rate of screw shaft 408 and piston 402 may be configured to negate the difference in the thermal expansion rate between the reservoir and the medicine.

The thermal expansion coefficients of each of reservoir chamber 220, dose dispensing piston 402, dose setting piston 404, and screw shaft 408 may be determined based on the materials used for each of these components. To obtain a desired combined thermal expansion rate of the set volume, a configuration including a particular combination of materials for the reservoir chamber 220, dose dispensing piston 402, dose setting piston 404, screw shaft 408, or any combination thereof, may be determined, and different configurations may be determined for different medicines.

As an illustrative example, for a medicine with a relatively small thermal expansion coefficient, reservoir chamber 220 and screw shaft 408 may be made of the same type of material (with identical thermal expansion coefficient). The dimension of screw shaft 408 may also be designed so that the net expansion by the shaft (which depends on the thermal expansion coefficient, the temperature difference, and the dimension) may compensate for the volumetric increase caused by the thermal expansion of reservoir chamber 220 (which enlarges cross-sectional area 602 and moves left edge 604 to the left). On the other hand, for a medicine with a relatively large thermal expansion coefficient, reservoir chamber 220 and screw shaft 408 may be made of different types of materials with a different thermal expansion coefficient, with reservoir chamber 220 being associated with a much larger thermal expansion coefficient than the one associated with screw shaft 408. Such arrangements may reduce the negative influence of screw shaft 408 on the change of the set volume, such that the set volume may increase in approximately the same rate (with respect to temperature) as the medicine.

In some embodiments, to simplify design, dose dispensing piston 402 and dose setting piston 404 may be constructed with materials that have relatively small thermal coefficients (compared with those of reservoir chamber 220 and screw shaft 408), so that the thermal expansion coefficients associated with reservoir chamber 220 and screw shaft 408 may dominate the combined thermal expansion coefficient and the combined rate of thermal expansion. Such arrangements may be used when, for example, medicine delivery device 110 is used for a small set of medicines, and the combined thermal expansion coefficient (and/or the combined rate of thermal expansion) of medicine delivery device 110 tracks a small set of thermal expansion coefficients of the small set of medicines. In some embodiments, dose dispensing piston 402 and dose setting piston 404 may also be designed to have relatively high thermal expansion coefficients (and rate of thermal expansion) to provide additional influences to the combined thermal expansion coefficient, such that the combined thermal expansion coefficient (and/or the combined rate of thermal expansion) tracks a larger set of thermal expansion coefficients of a larger set of medicines.

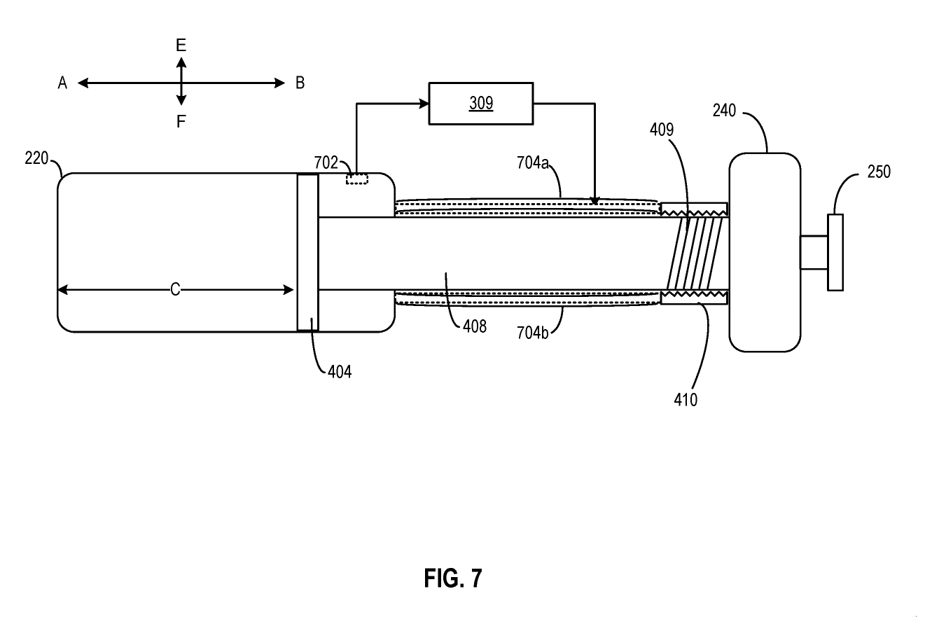

FIG. 7 illustrates another embodiment of medicine delivery device 110 for mitigating the impact of ambient conditions, according to aspects of the present disclosure. For illustration purposes, only some of the components of medicine delivery device 110 are shown in FIG. 7. As shown in FIG. 7, medicine delivery device 110 includes a sensor 702. Sensor 702 may be a pressure sensor configured to detect an ambient pressure inside reservoir chamber 220. Sensor 702 may also be a temperature sensor configured to detect an ambient temperature inside reservoir chamber 220. Although FIG. 7 shows that sensor 702 is located inside the reservoir chamber 220, it is understood that sensor 702 may also be external to reservoir to measure the ambient temperature and pressure information of the environment in which medicine delivery device 110 for deducing the ambient temperature and pressure inside reservoir chamber 220. Sensor 702 may also be submerged into the medicine held within reservoir chamber 220 to directly measure a physical state (e.g., temperature) of the medicine. Sensor 702 may be part of, for example, sensor(s) and actuator(s) 308 of FIG. 3.

In the embodiment of FIG. 7, medicine delivery device 110 further includes actuators 704a and 704b. Actuators 704a and 704b may include actuators made with certain materials of which a physical property (e.g., a size, a shape, etc.) may change based on a signal. For example, actuators 704a and 704b may be made of piezoelectric materials, shaped memory alloy (SMA), electroactive polymer, etc. The change in the physical property of actuators 704a and 704b may change distance C between the edges of reservoir chamber 220 and dose setting piston 404, which in turn changes the set volume of the medicine to be dispensed. For example, actuators 704a and 704b may be coupled with screw receiver 410 and with reservoir chamber 220. Actuators 704a and 704b may set or change a separation distance between screw receiver 410 and reservoir chamber 220. For example, actuators 704a and 704b may bend inward (towards directions F and E respectively, as indicated by the dotted lines), which causes screw receiver 410 to move to the right (towards direction B). Given that screw shaft 408 is also coupled with screw receiver 410, screw shaft 408, as well as dose setting piston 404, also move to the right. The movement increases distance C as well as the set volume of the medicine to be dispensed. On the other hand, if actuators 704a and 704b bend outward (towards directions E and F, respectively), the separation distance as well distance C may be decreased, which reduces the set volume of the medicine to be dispensed.

The operation of actuators 704a and 704b may be controlled by a processing circuit (e.g., hardware processor 309) based on the ambient condition data (or data indicating a physical condition of the medicine) provided by sensor 702. In some embodiments, actuators 704a and 704b, sensor 702, and hardware processor 309 may form a feedback loop to compensate for dosage error caused by volumetric changes of the medicine, reservoir chamber 220, dose dispensing piston 402, dose setting piston 404, screw shaft 408, etc. For example, in a case where the medicine expands in volume due to lower ambient pressure, hardware processor 309 may determine, based on the ambient pressure, the change in the volume of the medicine in reservoir chamber 220. To compensate for the volumetric change, hardware processor 309 may transmit a signal to actuators 704a and 704b to cause them to bend inward, so as to move dose setting piston 404 to the right (towards direction B), to increase the volume of the medicine to be dispensed. As another example, due to the uneven thermal expansions between the medicine and reservoir chamber 220, the level of the medicine inside reservoir chamber 220 drops (e.g., move towards direction A to the left). In that case, hardware processor 309 may transmit a signal to actuators 704a and 704b to cause them to bend outward, so as to move dose setting piston 404 to the left (towards direction A) as well, to track the change in the medicine level. In both cases, the locations of dose setting piston 404 may be adjusted to ensure that the same amount of molecules in a set dosage (indicated by the position of dose setting piston 404) to be administered remains the same across different ambient conditions.

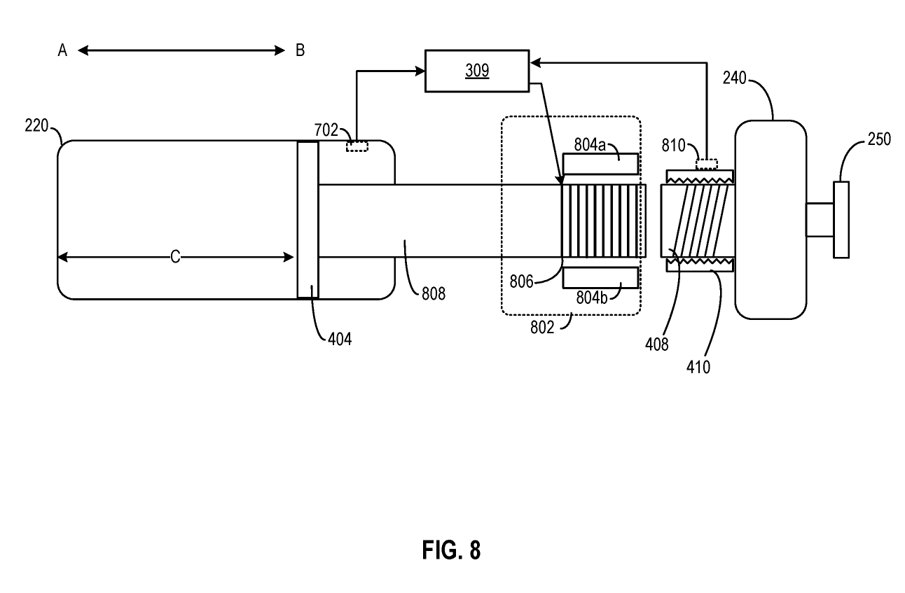

FIG. 8 illustrates another embodiment of medicine delivery device 110 for mitigating the impact of ambient conditions, according to aspects of the present disclosure. For illustration purposes, only some of the components of medicine delivery device 110 are shown in FIG. 8. As shown in FIG. 8, medicine delivery device 110 may include an electromagnetic assembly 802 comprising magnets 804a and 804b, as well as electric coil 806 that wraps around a shaft 808 that is coupled with dose setting piston 404. A processing circuit (e.g., hardware processor 309) may cause a current to flow through electric coil 806 which, combined with the magnetic fields generated by magnets 804a and 804b, may generate an electromagnetic force to move shaft 808 (and dose setting piston 404) towards directions A or B, to set a volume of the medicine to be dispensed.

Hardware processor 309 may determine the current to flow through electric coil 806 (and set the volume of the medicine to be dispensed) based on an input dosage as well as the ambient condition data provided by sensor 702. In the example of FIG. 8, medicine delivery device 110 includes a sensor 810 to sense a distance of movement of screw shaft 408 relative to screw receiver 410. The movement may be caused by the rotation of dose knob 240, and hardware processor 309 may interpret the distance of movement to represent an input dosage by the user. Hardware processor 309 may then determine the volume of the medicine to be displaced based on the input dosage, while compensating for the volumetric changes of the medicine, reservoir chamber 220, dose dispensing piston 402, dose setting piston 404, shaft 808, etc., as discussed above. Hardware processor 309 may also generate an output representing the compensated dosage, and provide the output to output interface 312.

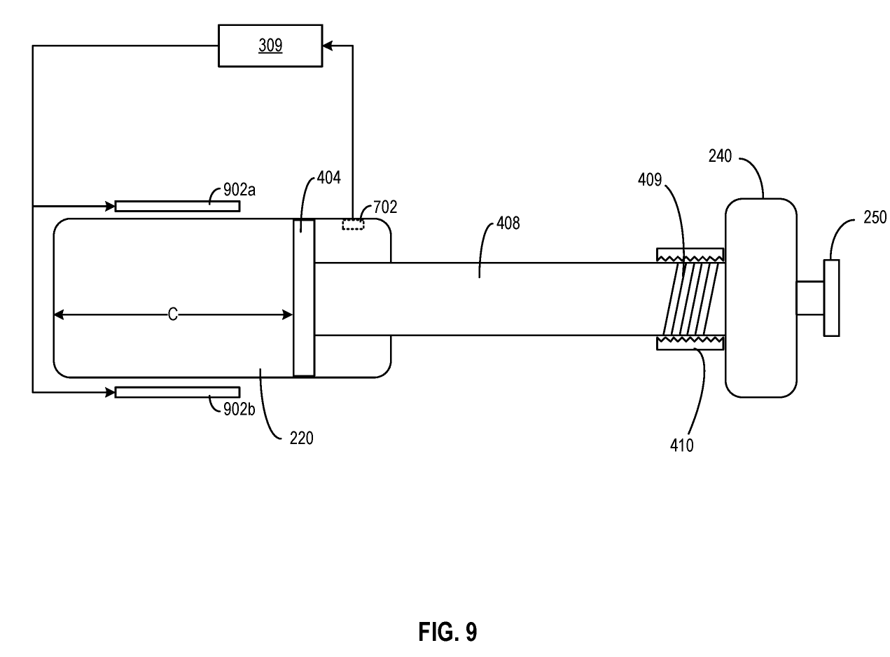

FIG. 9 illustrates another embodiment of medicine delivery device 110 for mitigating the impact of ambient conditions, according to aspects of the present disclosure. For illustration purposes, only some of the components of medicine delivery device 110 are shown in FIG. 9. As shown in FIG. 9, medicine delivery device 110 includes a pair of electrodes 902a and 902b coupled with a processing circuit (e.g., hardware processor 309) to generate an electric field over reservoir chamber 220. Reservoir chamber 220 may be made of electroactive polymer. As discussed above, a physical property (e.g., size, shape, etc.) of electroactive polymer may change with respect to an electric field applied across the polymer. Here, hardware processor 309 may change the electric field to effect a change in the volume of reservoir, to compensate for volumetric changes of the medicine, reservoir chamber 220, dose dispensing piston 402, dose setting piston 404, screw shaft 408, etc. For example, based on the ambient temperature information, hardware processor 309 may estimate the volumetric increases of the medicine as well as screw shaft 408, and may generate an electric field to expand the volume of reservoir chamber 220, so that the volume defined by reservoir chamber 220 and dose setting piston 404 (represented by distance C) may track the volume of the medicine across different ambient conditions.

Although not explicitly shown, it is understood that the techniques described in FIG. 7, FIG. 8, and FIG. 9 may be combined with the techniques of thermal expansion balancing described with respect to FIG. 6. For example, to counter the contract effect of the set volume caused by the thermal expansion of piston 404, the techniques described in FIG. 7, FIG. 8, and FIG. 9 may be used to adjust the position of piston 404 (e.g., by moving it backwards towards direction B) based on the ambient temperature, which may indicate a degree of thermal expansions of reservoir chamber 220, the medicine, and piston 404. Based on the degrees of thermal expansions, the position of piston 404 may be adjusted to counter the thermal expansion of the piston, such that the set volume expands according to the thermal expansion of reservoir chamber 220 and tracks the thermal expansion of the medicine.

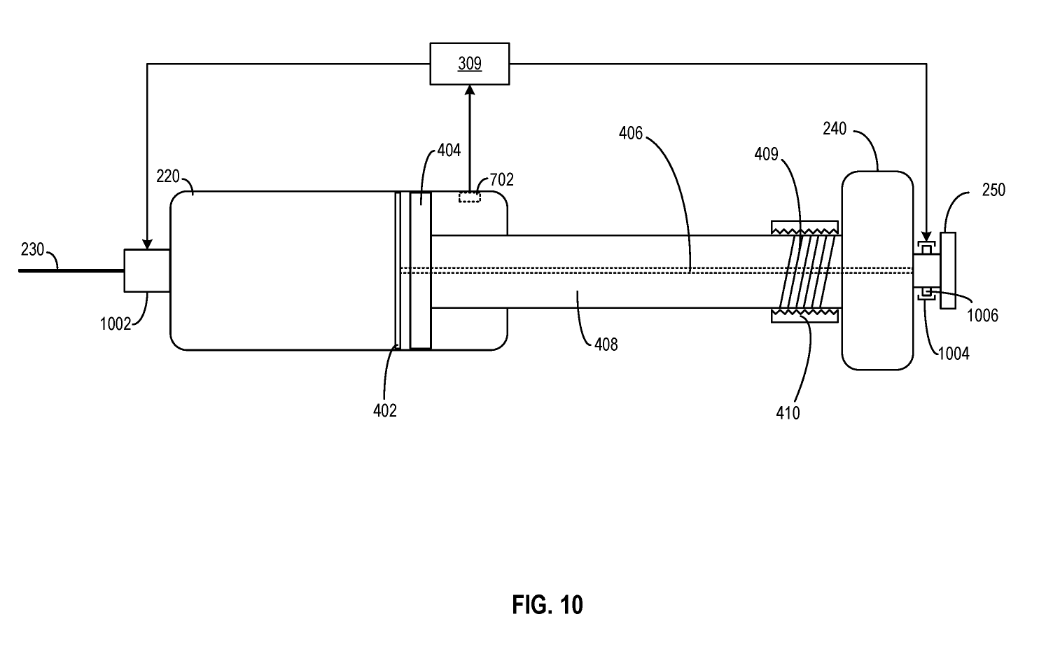

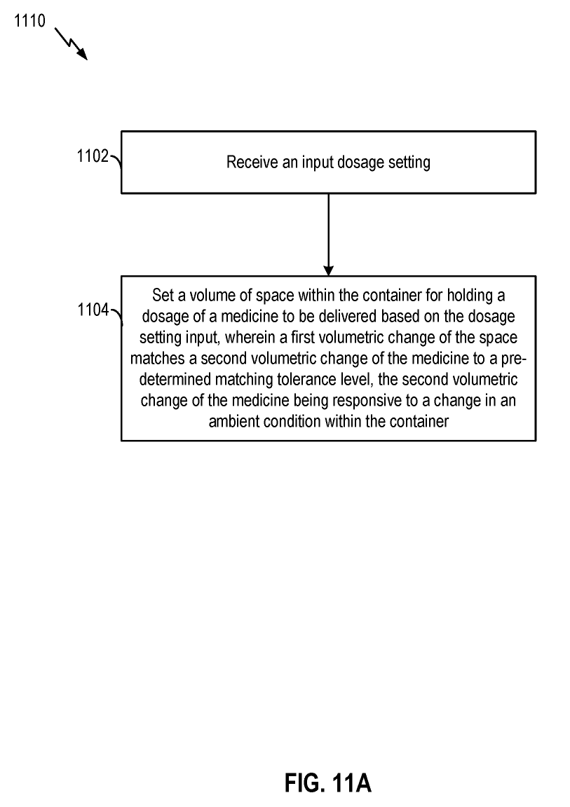

FIG. 10 illustrates another embodiment of medicine delivery device 110 for mitigating the impact of ambient temperature, according to certain aspects of the present disclosure. For illustration purposes, only some of the components of medicine delivery device 110 are shown in FIG. 10. As shown in FIG. 10, medicine delivery device 110 may include at least an electric valve 1002 between reservoir chamber 220 and needle assembly 230. Alternatively, or in combination with electric valve 1002, medicine delivery device 110 may further include a latch device 1004 that may latch with a protrusion structure 1006 of dose dispensing button 250. Electric valve 1002, latch device 1004, or any combination thereof may be controlled by a processing circuit (e.g., hardware processor 309) to disable dispensing of the medicine from reservoir chamber 220, based on the ambient condition of the medicine.

As discussed above, the physical state of the medicine may be affected by the ambient temperature. If the ambient temperature goes above a certain threshold, the chemical composition of the medicine may change, rendering the medicine ineffective or even unsafe for use. Here, hardware processor 309 may obtain the ambient temperature information inside reservoir chamber 220 from sensor 702, and determine whether the medicine stored in reservoir chamber 220 is suitable for administering to the user based on the temperature information. If the ambient temperature information exceeds a threshold, hardware processor 309 may, for example, transmit a signal to close electric valve 1002, to prevent the medicine from being dispensed through needle assembly 230. As another example, hardware processor 309 may also transmit a signal to cause latch device 1004 to latch onto protrusion structure 1006 of dose dispensing button 250, to lock dose dispensing button 250 as well as dose dispensing piston 402 at a fixed position. With such arrangements, hardware processor 309 may also prevent dose dispensing button 250 from pushing the medicine out of reservoir chamber 220 through needle assembly 230.

In some embodiments, before or after disabling the dispensing mechanism, hardware processor 309 may generate a message indicating that the temperature exceeds the threshold, and that the dispensing mechanism has been disabled as a result. Hardware processor 309 may output the message via output interface 312, and/or transmit the message to another device (e.g., connecting device 130) via communication interface 310. A user may determine to override the disabling mechanism and transmit, via another device (e.g., connecting device 130), an override signal back to medicine delivery device 110 to enable the dispensing mechanism. Alternatively, the system may also be reset (e.g., after replacing reservoir chamber 220, draining and refilling reservoir chamber 220 with new medicine, etc.) to enable the dispensing mechanism.

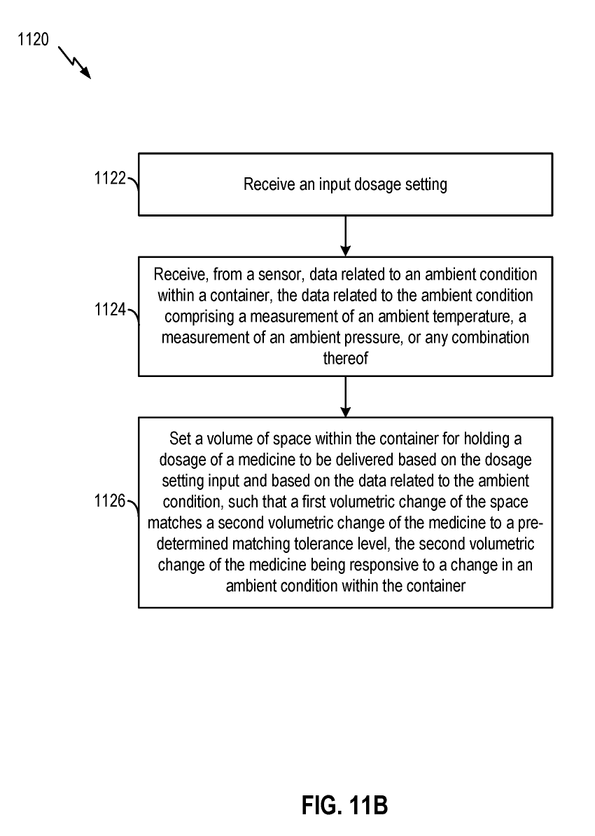

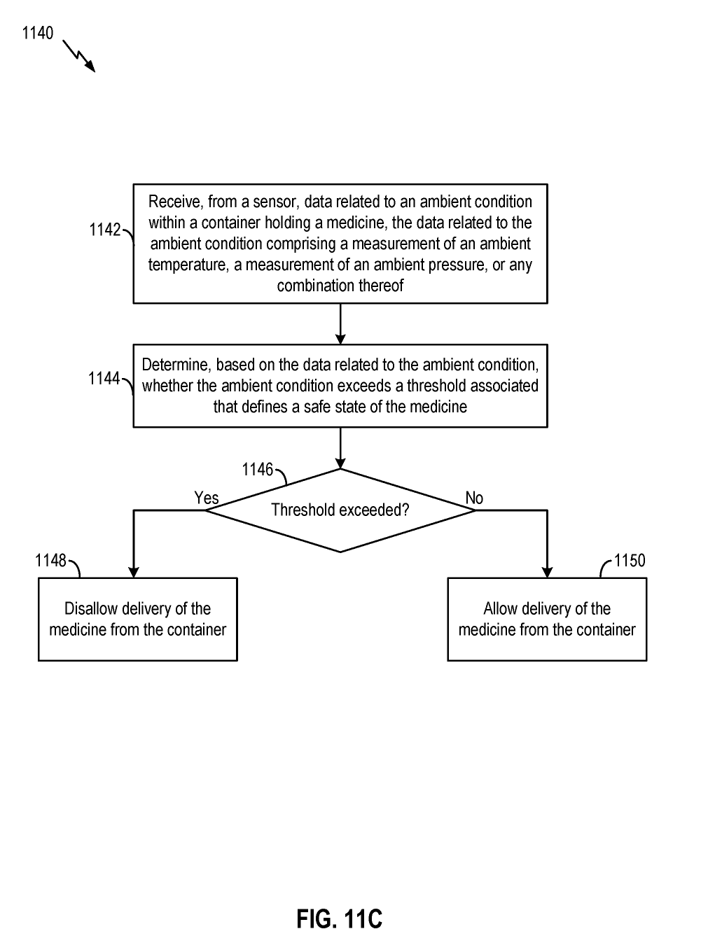

FIG. 11A, FIG. 11B, and FIG. 11C include flowcharts which illustrate examples of methods for facilitating administering of medicine according to certain aspects of the present disclosure. The process illustrated by the flowcharts of FIG. 11A, FIG. 11B, and FIG. 11C may be performed by various components of medicine delivery device 110 including, for example, reservoir chamber 220, dose control mechanism 304, dose dispensing mechanism 306, hardware processor 309, or any combination thereof.

FIG. 11A illustrates a flowchart 1110. Referring to FIG. 11A, at block 1102, the system may receive an input dosage setting to set a dosage of medicine to be delivered from a container (e.g., reservoir chamber 220). Means for performing the functionality of block 1102 may include, for example, dose control mechanism 304 comprising dose knob 240, dose selector 285, hardware processor 309, etc.

At block 1104, the system may set a volume of space within the container for holding a dosage of a medicine to be delivered based on the dosage setting input, wherein a first volumetric change of the space matches a second volumetric change of the medicine to a pre-determined matching tolerance level, the second volumetric change of the medicine being responsive to a change in an ambient condition within the container. The volumetric change can be caused by, for example, thermal expansion or contraction of the medicine responsive to a temperature change in the reservoir chamber 220, a pressure change in the reservoir chamber 220, or any combination thereof.

In some embodiments, at least one of the container or the dose control mechanism can be configured such that the thermal expansion/contraction of the space matches the thermal expansion/contraction of the medicine. For example, the container may have a first thermal expansion coefficient. The dosage control mechanism may have at least a second thermal expansion coefficient. The medicine may have a third thermal expansion coefficient. The first thermal expansion coefficient of the container and/or the second thermal expansion coefficient of the dosage setting structure may be determined based on the third thermal expansion coefficient of the medicine. For example, the first thermal expansion coefficient of the container and/or the second thermal expansion coefficient of the dosage control mechanism may be determined such that a first rate of thermal expansion or contraction of the space matches a second rate of thermal expansion or contraction of the medicine. In some embodiments, the medicine delivery device may include a sensor configured to collect data related to an ambient condition within the container, the data related to the ambient condition comprising a measurement of an ambient temperature, a measurement of an ambient pressure, or any combination thereof. The sensor may be configured to collected the data from an ambient environment within the container, from an environment external to the container, and/or from the medicine held in the container directly. The medicine delivery device may further include a processing circuit configured to control the container, the dosage control mechanism, or any combination thereof, to set the volume of the space based on the data related to the ambient condition. In some embodiments, the dosage control mechanism may include an actuator which may be controlled by the processing circuit to adjust the set volume based on the sensor data. In some embodiments, the container may be made of an electroactive polymer which may expand or contract based on an electric field from one or more electrodes, and the processing circuit may control the electric field based on the sensor data to adjust a volume of the container.

Means for performing the functionality of block 1104 may include components described in FIG. 6, FIG. 7, FIG. 8, and FIG. 9, and may include, for example, reservoir chamber 220, hardware processor 309, dispensing piston 402, dose setting piston 404, shaft 406, sensor 702, actuators 704a and 704b, magnets 804a and 804b, electric coil 806 electrodes 902a and 902b, etc.

FIG. 11B illustrates a flowchart 1120. Referring to FIG. 11B, at block 1122, the system may receive an input dosage setting to set a dosage of medicine to be delivered from a container (e.g., reservoir chamber 220). Means for performing the functionality of block 1102 may include, for example, dose control mechanism 304 comprising dose knob 240, dose selector 285, hardware processor 309, etc.

At block 1124, the system may receive, from a sensor, data related to an ambient condition within a container, the data related to the ambient condition comprising a measurement of an ambient temperature, a measurement of an ambient pressure, or any combination thereof. The ambient condition can be for an environment internal to reservoir chamber 220 or external to reservoir chamber 220. The ambient condition can also be measured based on directly measuring a physical condition (e.g., temperature) of the medicine. Means for performing the functionality of block 1124 may include components described in FIG. 7, FIG. 8, and FIG. 9 and may include, for example, sensor 702.

At block 1126, the system may set a volume of space within the container for holding a dosage of a medicine to be delivered based on the dosage setting input and based on the data related to the ambient condition, such that a first volumetric change of the space matches a second volumetric change of the medicine to a pre-determined matching tolerance level, the second volumetric change of the medicine being responsive to a change in an ambient condition within the container. In some embodiments, the system may include an actuator which may be controlled by the processing circuit to adjust the volume of the space (which is set based on the input dose setting) based on the sensor data by, for example, adjusting a position of dose setting piston 404 within reservoir chamber 220. In some embodiments, the container may be made of an electroactive polymer which may expand or contract based on an electric field from one or more electrodes, and the processing circuit may control the electric field based on the sensor data to stretch or contract the container to adjust the set volume.

Means for performing the functionality of block 1126 may include components described in FIG. 7, FIG. 8, and FIG. 9, and may include, for example, hardware processor 309, dispensing piston 402, dose setting piston 404, shaft 406, sensor 702, actuators 704a and 704b, magnets 804a and 804b, electric coil 806 electrodes 902a and 902b, etc.

FIG. 11C illustrates a flowchart 1140. Referring to FIG. 11C, at block 1142, the system may receive, from a sensor, data related to an ambient condition within a container, the data related to the ambient condition comprising a measurement of an ambient temperature, a measurement of an ambient pressure, or any combination thereof. The ambient condition can be for an environment internal to reservoir chamber 220 or external to reservoir chamber 220. The ambient condition can also be measured based on directly measuring a physical condition (e.g., temperature) of the medicine. Means for performing the functionality of block 1124 may include components described in FIG. 7, FIG. 8, FIG. 9, and FIG. 10, and may include, for example, sensor 702.

At block 1144, the system may determine, based on the data related to the ambient condition, whether the ambient condition exceeds a threshold associated that defines a safe state of the medicine. For example, the medicine may have a temperature threshold above which the medicine may perish or otherwise become unsafe for consumption. The system may determine the ambient temperature (and/or the temperature of the medicine held within the container) based on the sensor data, and determine whether the temperature exceeds a temperature threshold. Means for performing the functionality of block 1124 may include components described in FIG. 7, FIG. 8, FIG. 9, and FIG. 10, and may include, for example, hardware processor 309.

If the system determines, at block 1146, that the threshold is exceeded, the system may proceed to disallow delivery of the medicine from the container, at block 1148. If the threshold is not exceeded (at block 1146), the system may proceed to allow delivery of the medicine from the container, at block 1150. The system may disallow/allow delivery of the medicine from the container based on locking (or unlocking) the dose dispensing mechanism 306, which may include, for example, controlling latch device 1004 to latch with (or unlatch from) a protrusion structure 1006 of dose dispensing button 250, closing (or opening) electric valve 1002, etc., to prevent (or to allow) the medicine from leaving reservoir chamber 220 via needle assembly 230. Means for performing the functionality of blocks 1148 and 1150 may include components described in FIG. 7, FIG. 8, FIG. 9, and FIG. 10, and may include, for example, latch device 1004, protrusion structure 1006, dose dispensing button 250, electric valve 1002, etc.

The methods, systems, and devices discussed above are examples. Various configurations may omit, substitute, or add various procedures or components as appropriate. For instance, in alternative configurations, the methods may be performed in an order different from that described, and/or various stages may be added, omitted, and/or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the spirit or scope of the disclosure.

Also, configurations may be described as a process which is depicted as a flow diagram or block diagram. Although each may describe the operations as a sequential process, many of the operations may be performed in parallel or concurrently. In addition, the order of the operations may be rearranged. A process may have additional steps not included in the figure. Furthermore, examples of the methods may be implemented by hardware, software, firmware, middleware, microcode, hardware description languages, or any combination thereof. When implemented in software, firmware, middleware, or microcode, the program code or code segments to perform the necessary tasks may be stored in a non-transitory computer-readable medium such as a storage medium. Processors may perform the described tasks.

Having described several example configurations, various modifications, alternative constructions, and equivalents may be used without departing from the spirit of the disclosure. For example, the above elements may be components of a larger system, wherein other rules may take precedence over or otherwise modify the application of the invention. Also, a number of steps may be undertaken before, during, or after the above elements are considered.

1. A medicine delivery device, comprising:
a container; and
a dosage setting structure configured to:
receive a dosage setting input; and
set a volume of space within the container for holding a dosage of a medicine to be delivered based on the dosage setting input,
wherein the container, the dosage setting structure, or any combination thereof, is configured such that a first volumetric change of the space matches a second volumetric change of the medicine in the container to within a pre-determined matching tolerance level.

2. The medicine delivery device of claim 1, wherein the first volumetric change and the second volumetric change are due to thermal expansion or thermal contraction;
wherein the container has a first thermal expansion coefficient;
wherein the dosage setting structure has at least a second thermal expansion coefficient;
wherein the medicine has a third thermal expansion coefficient; and
wherein at least one of the first thermal expansion coefficient or the second thermal expansion coefficient is related to the third thermal expansion coefficient such that the first volumetric change and the second volumetric change, both of which are responsive to a change in an ambient temperature within the container, match to within the pre-determined matching tolerance level.

3. The medicine delivery device of claim 2, wherein the first thermal expansion coefficient, the second thermal expansion coefficient, the third thermal expansion coefficient, or any combination thereof, is negative.

4. The medicine delivery device of claim 2, wherein the dosage setting structure includes a shaft and a piston;
wherein the shaft has the second thermal expansion coefficient; and
wherein the piston has a fourth thermal expansion coefficient of opposite polarity to the second thermal expansion coefficient.

5. The medicine delivery device of claim 4, wherein a relationship between an absolute value of the second thermal expansion coefficient and an absolute value of the fourth thermal expansion coefficient reflects a relationship between one or more dimensions of the shaft and the piston such that a volumetric change of the shaft and a volumetric change of the piston, both of which responsive to the change in the ambient temperature within the container, substantially cancel out each other; and
wherein the one or more dimensions comprise: a length, a width, a cross-sectional area, a volume, or any combination thereof.

6. The medicine delivery device of claim 4, wherein the first thermal expansion coefficient and the third thermal expansion coefficient are equal.

7. The medicine delivery device of claim 4, wherein the piston and the shaft comprise different materials.

8. An apparatus, comprising:
means for holding a medicine;
means for receiving a dosage setting input; and
means for setting a volume of space within the means for holding the medicine, the volume of space for holding a dosage of a medicine to be delivered based on the means for receiving the dosage setting input,
wherein the means for holding the medicine, the means for receiving the dosage setting input, or any combination thereof, is configured such that a first volumetric change of the space matches a second volumetric change of the medicine to within a pre-determined matching tolerance level.

9. The apparatus of claim 8, wherein the first volumetric change and the second volumetric change are due to thermal expansion or thermal contraction;
wherein the means for holding the medicine has a first thermal expansion coefficient;
wherein the means for setting the volume of space within the means for holding the medicine has at least a second thermal expansion coefficient;
wherein the medicine has a third thermal expansion coefficient; and
wherein at least one of the first thermal expansion coefficient or the second thermal expansion coefficient is related to the third thermal expansion coefficient such that the first volumetric change and the second volumetric change, both of which are responsive to a change in an ambient temperature within the means for holding the medicine, match to within the pre-determined matching tolerance level.

10. The apparatus of claim 9, wherein the first thermal expansion coefficient, the second thermal expansion coefficient, the third thermal expansion coefficient, or any combination thereof, is negative.

11. The apparatus of claim 9, wherein the means for setting the volume of space within the means for holding the medicine includes a shaft and a piston;
wherein the shaft has the second thermal expansion coefficient; and
wherein the piston has a fourth thermal expansion coefficient of opposite polarity to the second thermal expansion coefficient.

12. The apparatus of claim 11, wherein a relationship between an absolute value of the second thermal expansion coefficient and an absolute value of the fourth thermal expansion coefficient reflects a relationship between one or more dimensions of the shaft and the piston such that a volumetric change of the shaft and a volumetric change of the piston, both of which responsive to the change in the ambient temperature within the means for setting the volume of space within the means for holding the medicine, substantially cancel out each other; and
wherein the one or more dimensions comprise: a length, a width, a cross-sectional area, a volume, or any combination thereof.

13. The apparatus of claim 11, wherein the first thermal expansion coefficient and the third thermal expansion coefficient are equal.

14. The apparatus of claim 11, wherein the piston and the shaft comprise different materials.

15. A method, comprising:
receiving, by a dosage setting structure coupled with a container, an input dosage setting, the dosage setting structure and the container being part of a medicine delivery device; and
setting, by the dosage setting structure in conjunction with the container, a volume of space within the container for holding a dosage of a medicine to be delivered based on the dosage setting input,
wherein a first volumetric change of the space matches a second volumetric change of the medicine in the container responsive to a change in an ambient condition to a pre-determined matching tolerance level.

16. The method of claim 15, wherein the first volumetric change and the second volumetric change are due to thermal expansion or thermal contraction;
wherein the container has a first thermal expansion coefficient;
wherein the dosage setting structure has at least a second thermal expansion coefficient;
wherein the medicine has a third thermal expansion coefficient; and
wherein at least one of the first thermal expansion coefficient or the second thermal expansion coefficient is determined based on the third thermal expansion.

17. The method of claim 16, wherein the first thermal expansion coefficient, the second thermal expansion coefficient, the third thermal expansion coefficient, or any combination thereof, is negative.

18. The method of claim 16, wherein the dosage setting structure includes a shaft and a piston;
wherein the shaft has the second thermal expansion coefficient; and
wherein the piston has a fourth thermal expansion coefficient of opposite polarity to the second thermal expansion coefficient.

19. The method of claim 18, wherein a relationship between an absolute value of the second thermal expansion coefficient and an absolute value of the fourth thermal expansion coefficient reflects a relationship between one or more dimensions of the shaft and the piston such that a volumetric change of the shaft and a volumetric change of the piston, both of which responsive to the change in the ambient temperature within the container, substantially cancel out each other; and
wherein the one or more dimensions comprise: a length, a width, a cross-sectional area, a volume, or any combination thereof.

20. The medicine delivery device of claim 18, wherein the first thermal expansion coefficient and the third thermal expansion coefficient are equal.
